(19) United States
(12) Patent Application Publication
(10) Pub. No.: US 2019/0365300 A1
(43) Pub. Date: Dec. 5, 2019

(54) EVALUATION OF AN AMOUNT OF A SUBSTANCE CONTAINED WITHIN CIRCULATING BLOOD

(71) Applicant: ZEDSEN LIMITED, LONDON (GB)

(72) Inventor: Hrand Mami MAMIGONIANS, London (GB)

(21) Appl. No.: 16/421,930

(22) Filed: May 24, 2019

(30) Foreign Application Priority Data
May 31, 2018 (GB) 1808857.5
Apr. 27, 2019 (GB) 1905972.4
May 4, 2019 (GB) 1906386.6

(51) Int. Cl. A61B 5/145

(52) U.S. Cl. CPC A61B 5/14532; G01L 1/142; A61B 5/14546

(57) ABSTRACT

An amount of a substance contained within blood circulating within the human body tissue is evaluated. An application region of an apparatus makes skin contact at the position of the tissue. The apparatus includes electrodes arranged on a dielectric substrate to present an active surface at the application region. Force data is derived from a detector, that is compared against a first predetermined level 1401. In response to this comparing step, capacitive coupling procedures are performed or these procedures are inhibited. Thus, procedures are inhibited if a sufficient force has not been applied. If a sufficient force has been applied, capacitive coupling procedures capacitively couple selected pairs of the electrodes by producing electric fields that penetrate the tissue, to produce monitored output data, from which the evaluation is made.

### CROSS REFERENCE TO RELATED APPLICATIONS

This application claims priority from United Kingdom patent application number 1808857.5, filed May 31, 2018; United Kingdom patent application number 1905972.4 filed Apr. 27, 2019 and United Kingdom patent application number 1906386.6 filed May 4, 2019. The whole content of United Kingdom patent application number 1808857.5, United Kingdom patent application number 1905972.4, and United Kingdom patent application number 1906386.6 is incorporated herein by reference.

### BACKGROUND OF THE INVENTION

The present invention relates to an apparatus for the non-invasive evaluation of an amount of a substance contained within blood circulating within human body tissue. The present invention also relates to a method of determining an amount of a substance contained within blood circulating within human body tissue.

It is known to examine objects using electric fields, as described in U.S. Pat. No. 8,994,383 assigned to the present applicant. However, problems arise when examining objects that are not homogeneous, such as biological tissues.

It is also known to adjust the penetration of electric fields by selecting different combinations of transmitter electrode and receiver electrode, with different separation distances. Thus, as the distance between a transmitter electrode and a receiver electrode increases, a greater degree of penetration is achieved.

A further problem arises in terms of evaluating an amount of a substance (such as glucose) contained within blood circulating within human body tissue. In particular, it is necessary for a subject to establish skin contact with insulated electrodes positioned on a dielectric substrate. Experiment has shown that fingers provide good candidates for making evaluations of this type, given the relatively high concentration of capillaries close to the skin. However, experiments also suggest that measurements may be influenced by changes in temperature, humidity and applied pressure. In particular, it is necessary for a sufficient level of pressure to be applied in order to achieve satisfactory results via the capacitive coupling of displaced electrodes.

### BRIEF SUMMARY OF THE INVENTION

According to a first aspect of the present invention, there is provided an apparatus for the non-invasive evaluation of an amount of a substance contained within blood circulating within human body tissue, comprising: an application region arranged to make skin contact; a plurality of electrodes arranged on a dielectric substrate, thereby presenting an active surface at the position of said application region; a detector configured to produce force data representing an applied force or pressure upon said active surface; and a processor, wherein said processor configured to: compare said force data against a first predetermined level; perform capacitive-coupling procedures that capacitively couple selected pairs of said electrodes by producing electric fields that penetrate said tissue to produce monitored output data, if said force data is higher than said first predetermined level; and inhibit said capacitive-coupling procedures if said force data is not above said first predetermined level.

In an embodiment, the apparatus includes a display device configured to indicate that an increase in applied force/pressure is required if the capacitive coupling procedures have been inhibited.

In an embodiment, the processor is also configured to inhibit the capacitive coupling procedures if the force data is above a second predetermined level. The apparatus may also include a display device configured to indicate that a reduction in applied force/pressure is required.

In an embodiment, the processor is also configured to store force data when the capacitive coupling procedures are performed.

According to a second aspect of the present invention, there is provided a method of determining an amount of a component contained within blood circulating within human body tissue, comprising the steps of: locating an application region of an apparatus to make skin contact at the position of said tissue, wherein said apparatus includes a plurality of electrodes arranged on a dielectric substrate to present an active surface at said application region; deriving force data from a detector; comparing said force data, representing applied force/pressure of said skin contact upon said application region, against a first predetermined level; and in response to said comparing step, performing a step selected from a list consisting of: performing capacitive-coupling procedures that capacitively couple selected pairs of said electrodes by producing electric fields that penetrate said tissue to produce monitored output data, if sufficient force has been applied; and inhibiting said capacitive-coupling procedures if a sufficient force has not been applied.

Embodiments of the invention will be described, by way of example only, with reference to the accompanying drawings. The detailed embodiments show the best mode known to the inventor and provide support for the invention as claimed. However, they are only exemplary and should not be used to interpret or limit the scope of the claims. Their purpose is to provide a teaching to those skilled in the art. Components and processes distinguished by ordinal phrases such as “first” and “second” do not necessarily define an order or ranking of any sort.

### BRIEF DESCRIPTION OF THE SEVERAL VIEWS OF THE DRAWINGS

FIG. 1 shows a pressure-compensating non-invasive blood-containing substance measuring apparatus;

FIG. 2 shows a main circuit board of the apparatus of FIG. 1;

FIG. 3 shows the underside of the main circuit board identified in FIG. 2;

FIG. 4 shows a cross section of the apparatus of FIG. 1;

FIG. 5 details a force sensor of the type shown in FIG. 4;

FIG. 6 shows a schematic representation of the apparatus shown in FIG. 1;

FIG. 7 shows a schematic representation of an energizing circuit, of the type identified in FIG. 6;

FIG. 8 shows an example of the multiplexing environment identified in FIG. 6;

FIG. 9 illustrates two mutually orthogonal electrode groups;

FIG. 10 shows an example of the analog processing circuit identified in FIG. 6;

FIG. 11 shows procedures performed by the processor identified in FIG. 6;

FIG. 12 shows an example of a scan cycle;

FIG. 13 illustrates a message inviting a subject to deploy a finger;

FIG. 14 shows examples of applied force during an evaluation period;

FIG. 15 illustrates a second message to a subject confirming that scanning operations are being performed;

FIG. 16 illustrates a third message to a subject inviting them to press harder;

FIG. 17 illustrates a fourth message to a subject inviting them to press less hard;

FIG. 18 illustrates an output data block;

FIG. 19 illustrates operations performed by a machine learning system;

FIG. 20 shows a pattern of electric fields generated from a plurality of electrodes by an embodiment;

FIG. 21 shows the relationship between an energizing input pulse and a monitored output signal;

FIG. 22 illustrates an embodiment for achieving the pattern identified in FIG. 20, in which a first electrode is energized;

FIG. 23 shows a common electrode data set produced from the coupling operations shown in FIG. 22;

FIG. 24 illustrates the energization of a second electrode;

FIG. 25 illustrates a common electrode data set produced from the coupling operations illustrated in FIG. 24;

FIG. 26 continues to illustrate a process of sequentially selecting adjacent electrodes as electrodes in common;

FIG. 27 illustrates data produced from the energization of electrode 14;

FIG. 28 illustrates the start of reverse layering from electrode 15;

FIG. 29 shows a common electrode data set by reverse layering;

FIG. 30 shows a layering data set;

FIG. 31 shows a third stage of operation, inviting the finger to be removed; and

FIG. 32 shows a fourth stage of operation, presenting an indication of glucose level.

### DETAILED DESCRIPTION OF EMBODIMENTS OF THE INVENTION

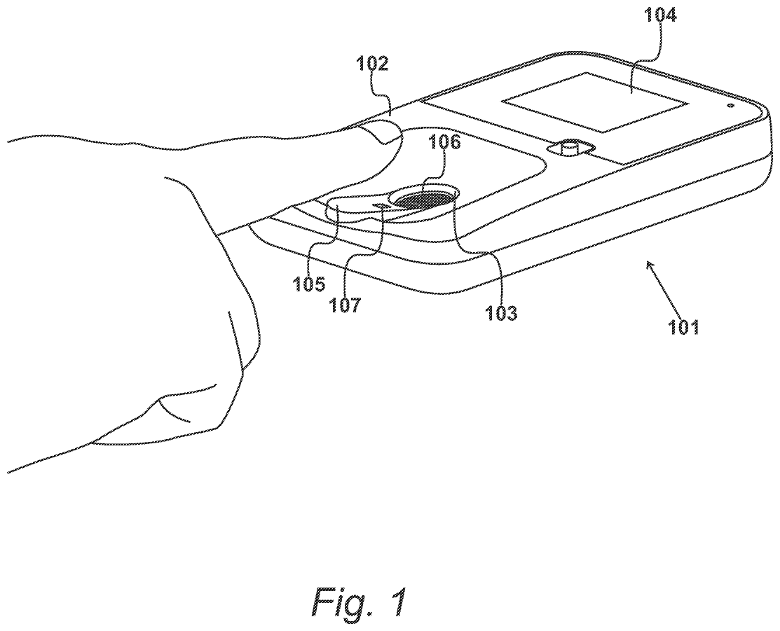

### FIG. 1

An apparatus 101 for the non-invasive evaluation of an amount of a substance contained within human body tissue is shown in FIG. 1. The apparatus has a plastic housing 102, with a membrane-exposing orifice 103, presenting an application region arranged to make skin contact. In this embodiment, contact is made against the skin of a finger, but in other embodiments, contact can be made with other suitable areas of the body, such as a wrist.

The housing also includes a visual display orifice 104 which, in this embodiment, is covered by a transparent cover, thereby allowing a visual display unit, supported by a main circuit board, to be seen during the operation of the apparatus. In this embodiment, the visual display unit is a liquid crystal display but other types of display can be deployed. In alternative embodiments, the display may take the form of devices configured to emit light of various colors. Alternatively, a display may be presented to a subject (the person being tested) or to an operative (such as a clinician) via an alternative device, such as a wireless connected mobile device.

A guide portion 105 guides a subject's finger into position, to contact with an electrode supporting membrane 106. The guide portion 104 also includes a temperature sensor 107. An additional temperature sensor and a humidity sensor may also be included within the housing. In this way, each data set produced during a scanning operation may include temperature data and humidity data in addition to data representing a degree of applied force or pressure.

In this embodiment, a device measures applied force thereby allowing a processor to compare force data against a predetermined level. A minimum level of pressure is required to ensure that a reliable contact is made between the subject's finger and the electrode supporting membrane 106. Thus, testing is inhibited if the force data is not above this predetermined level.

The electrodes are coated with a thin layer of an insulating material, such that an applied finger does not make electrical contact with the electrodes but does capacitively engage with the electrodes; such that it is possible for electric fields to enter the fingertip without an airgap being present.

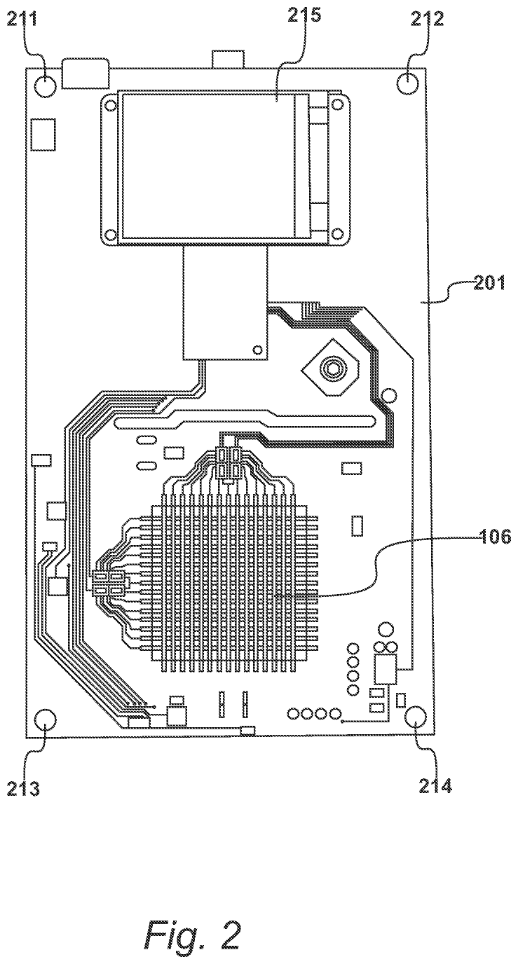

### FIG. 2

A main circuit board 201 is shown in FIG. 2, upon which the electrode supporting membrane 106 is itself supported above a first orifice. In this way, when pressure is applied to the membrane 106, a limited degree of movement is possible, resulting in force being applied to a force sensor.

A plurality of electrically insulated substantially parallel electrodes are mounted on the dielectric membrane 106 and the main circuit board 201 provides electrical connections to these electrodes. In this embodiment, in addition to a first group of substantially parallel electrodes mounted on the top surface of the dielectric membrane, a second group of substantially parallel electrodes are mounted on the underside of the dielectric membrane 106.

The orientation of the second group of electrodes is offset with respect to the orientation of the first group of electrodes. In this embodiment, the first group of electrodes are mutually orthogonal to the second group of electrodes. In this way, it is possible for a first layering operation to be performed using the first group, followed by a second layering operation being performed using the second group. Conventional position detection is also possible by sequentially energizing electrodes of one of these groups while monitoring selected electrodes of the other group.

Circuit board 201 is secured to the housing 102 at a first securing location 211, a second securing location 212, a third securing location 213 and a fourth securing location 214. A visual display unit 215 is attached to the main circuit board 201.

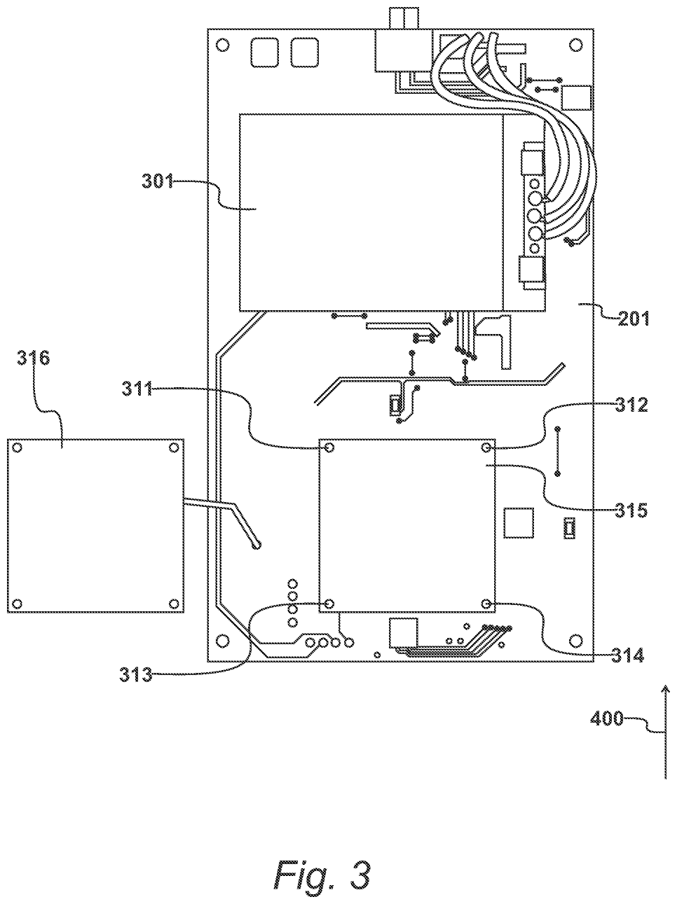

### FIG. 3

The underside of the main circuit board 201 is shown in FIG. 3. A rechargeable battery 301 provides electrical power for components within the apparatus. Plural fixing elements, consisting of a first rod 311, a second rod 312, a third rod 313 and a fourth rod 314 are secured to the main circuit board 201. In an embodiment, these fixing elements (rods) are secured by being soldered to circuit board 201. In an embodiment, a plastic support 315 is located on rods 311 to 314 to support the dielectric membrane 106. In an embodiment, the plastic support is derived from an acetyl material, selected such that electrical properties of this material do not change with respect to changes in temperature and humidity experienced within the operational environment.

Following the application of support 315, an intermediate board 316 is deployed over the rods 311 to 314, such that it is guided but not restrained by these fixing elements. In this way, board 316 is allowed to move and as such applies force onto the force sensor. In an embodiment, the intermediate board 316 includes an electrically conductive ground plane to provide electrical shielding to the lower side of the membrane 106.

After deploying the intermediate board 316, a bottom circuit board is located on the fixing elements 311 to 314 and thereafter secured to the fixing elements. Thus, the plural fixing elements secure the bottom circuit board to the top circuit board, such that the bottom circuit board does not move with respect to the main circuit board and the bottom circuit board does not contact the housing 102 directly.

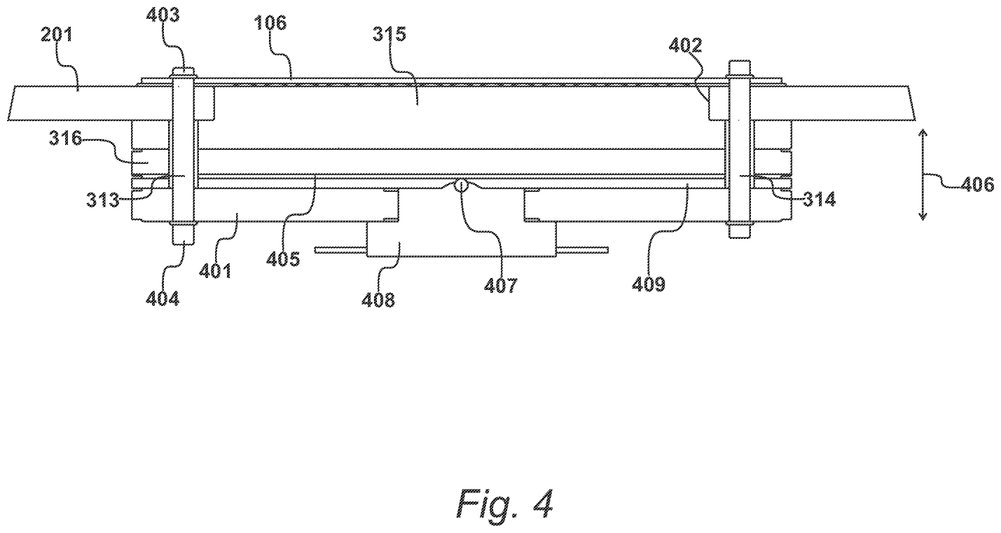

A cross-sectional view of the apparatus, looking in the direction of arrow 400, is shown in FIG. 4.

### FIG. 4

Metal rod 313 and metal rod 314 are shown in the cross section of FIG. 4. These, along with the other two fixing rods, secure a bottom circuit board 401 to the top circuit board 201. The top circuit board 201 includes a first orifice 402 and the dielectric membrane 106 is supported over this orifice. In an embodiment, the membrane has a thickness of typically 0.1 millimetres and the main circuit board 201 has a typical thickness of 1.6 millimetres. Each of the metal rods, including metal rod 313, has an upper end 403 and a lower end 404 such that, in an embodiment, the upper ends 403 are soldered to the main circuit board 201 and the lower ends 404 are soldered to the bottom circuit board 401.

The acetyl support 315 is shown in FIG. 4, along with the intermediate board 316 with a ground plane 405. Support 315 and board 316 are guided by the fixing elements 311 to 314 but are not restrained by these fixing elements, such that they are free to move in a vertical direction, as indicated by arrow 406. Relatively little movement may occur, typically up to a maximum of ten micrometres but it is unlikely that this would be perceived by a subject. Movement is restrained by a metal ball 407 extending from a force sensor 408, wherein the metal ball 407 is in contact with the ground plane 405 attached intermediate board 316.

In this embodiment, the force sensor 408 is received within an orifice provided within the bottom circuit board 401, with the metal ball 407 extending above the plane of the bottom circuit board 401. Thus, in this way, an extending portion of the force sensor extends above a top surface of the bottom circuit board.

In an embodiment, the extending portion is surrounded by an elastomeric material 409. In an embodiment, the elastomeric material is a silicone rubber with a Shore durometer (type A) of less than forty. Thus, when flexing occurs, due to applied pressure, the elastomeric material 409 compresses. Thereafter, when force is removed, the elastomeric material will expand back to its original position, thereby ensuring that the apparatus is returned to a fully operational state.

Thus, in an embodiment, a subassembly is formed consisting of the bottom circuit board 401 and an inserted force sensor 408 with an extending portion surrounded by the elastomeric material 409. This subassembly is then located over the fixing elements and soldered into position, as previously described.

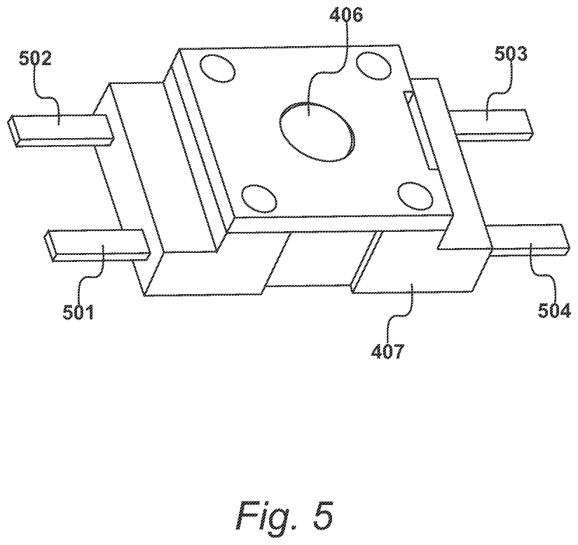

### FIG. 5

An example of force sensor 405 is illustrated in FIG. 5, that may be an FSS low profile force sensor produced by Honeywell Corporation, Illinois USA. As illustrated in FIG. 5, the metal ball (of stainless steel) is located at the centre of the sensor. Internally, the sensor uses a piezo-resistive micro-machined silicone sensing element. The sensor is configured such that its resistance will increase when the sensing element flexes under an applied force. The stainless-steel ball 406 concentrates this force directly onto the silicon-sensing element. Thus, resistance changes in proportion to the amount of force being applied.

The device presents a first pin 501, a second pin 502, a third pin 503 and a fourth pin 504. Up to a maximum of twelve volts is applied across the first pin 501 and the third pin 503. Sensor output is then measured as a differential voltage across the second pin 502 and the fourth pin 504. Other types of sensor may be deployed, such as those directly measuring pressure or strain.

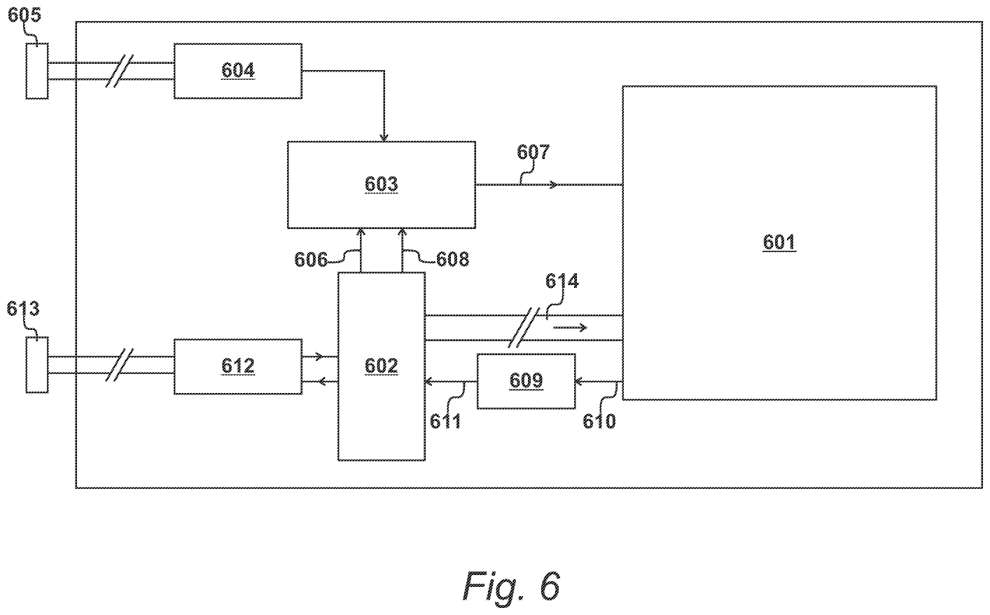

### FIG. 6

A schematic representation of an examination apparatus embodying the present invention is shown in FIG. 4. A multiplexing environment 601 includes a dielectric membrane supporting at least one group of parallel electrodes. Environment 601 also includes a de-multiplexer for de-multiplexing energizing input pulses, along with a multiplexer for combining selected output signals.

A processor 602, implemented as a microcontroller, addresses the de-multiplexer and the multiplexer to ensure that the same electrode cannot be both energized as a transmitter and monitored as a receiver during the same coupling operation. An energizing circuit 603 is energized by a power supply 604 that may in turn receive power from an external source via a power input connector 605. A voltage-control line 606 from the processor 602 to the energizing circuit 603, allows processor 602 to control the voltage (and hence the energy) of the energizing signals supplied to the multiplexing environment via a strobing line 607. The timing of each energizing signal is controlled by the processor 602 via a trigger-signal line 608.

An output from the multiplexing environment 601 is supplied to an analog-processing circuit 609 over a first analog line 610. A conditioning operation is performed by the analog-processing circuit 609, allowing analog output signals to be supplied to the processor 602 via a second analog line 611. The processor 602 also communicates with a two-way-data-communication circuit 612 to facilitate the connection of a data-communication cable. In an alternative embodiment, communication with external devices is achieved using a wireless protocol.

During scanning operations, the processor 602 supplies addresses over address buses 614 to the multiplexing environment 601, to define a pair of capacitively coupled electrodes. An energization operation is performed by applying an energizing voltage to strobing line 607, monitoring a resulting output signal and sampling the output signal multiple times to capture data indicative of a peak value and a rate of decay.

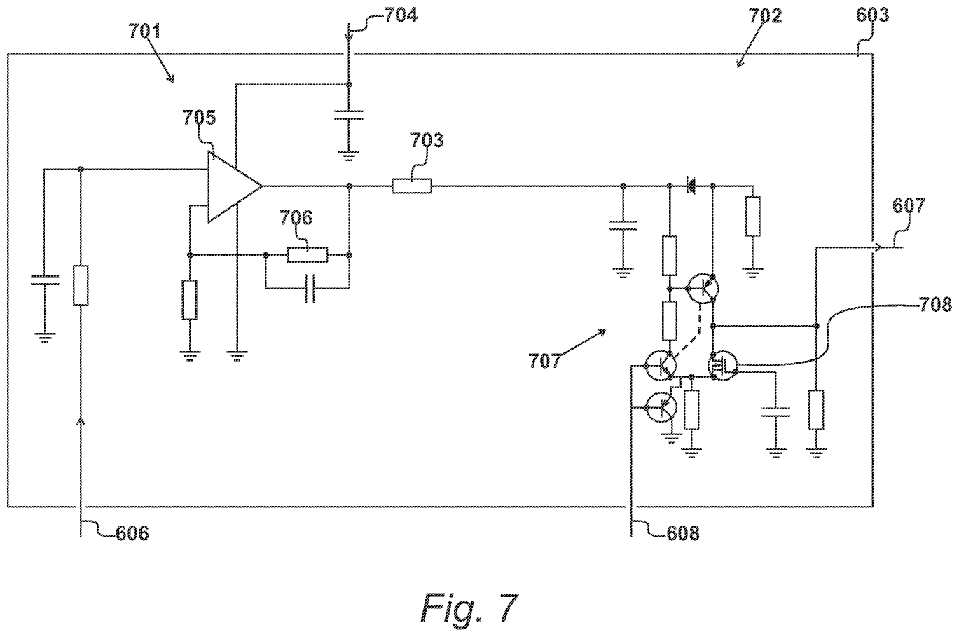

### FIG. 7

A schematic representation of the energizing circuit 603 is shown in FIG. 7. The energizing circuit includes a voltage-control circuit 701 connected to a strobing circuit 702 via current-limiting resistor 703. A voltage-input line 704 receives energizing power from the power supply to energize an operational amplifier 705. Amplifier 705 is configured as a comparator and receives a reference voltage via a feedback resistor 706. This is compared against a voltage-control signal received on the voltage-control line 606, to produce an input voltage for the strobing circuit 702.

The strobing circuit includes two bipolar transistors configured as a Darlington pair 707, in combination with a MOSFET (metal oxide silicon field effect transistor) 708. This creates energizing pulses with sharp rising edges, that are conveyed to the strobing line 607, after receiving a trigger signal on line 608.

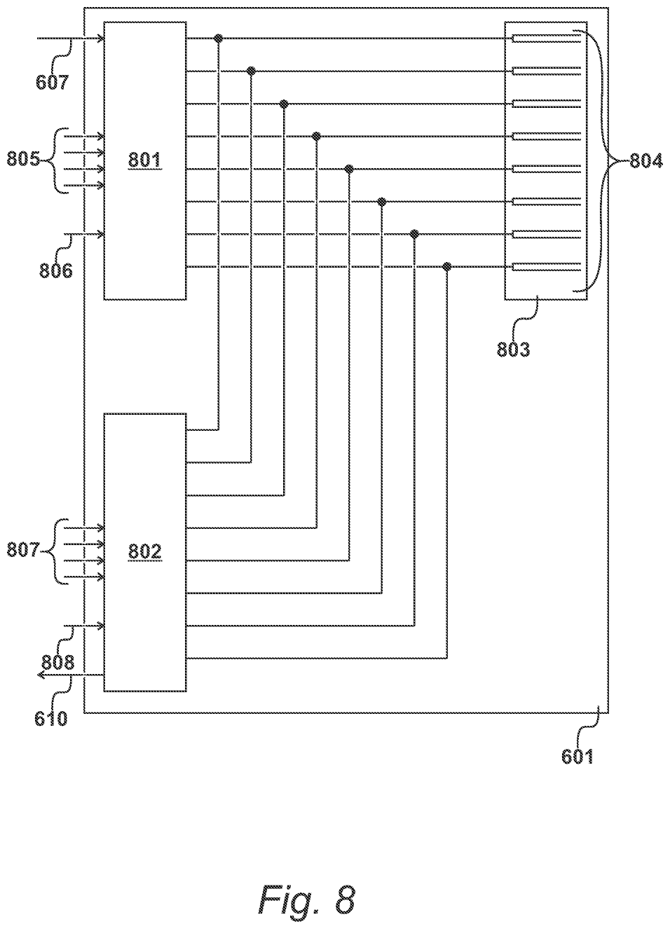

### FIG. 8

An example of a multiplexing environment is shown in FIG. 8, in which a first multiplexing device 801 supplies energizing input signals to a selected transmitter electrode, while a second multiplexing device 802 monitors output signals from a selected receiver electrode. A dielectric membrane 803 supports plural parallel electrodes coated with an insulating coating to allow them to be brought into contact with the subject's finger. Eight linear electrodes 804 are shown for illustrative purposes but more or fewer electrodes may be included.

An embodiment will be described in greater detail that includes two mutually offset groups, with fifteen electrodes in each group. Such an arrangement facilitates measurement in two dimensions; with the second group of electrodes being provided with respective multiplexing devices.

The first multiplexing device 601 includes first address lines 605 and an enabling line 606. Similarly, the second multiplexing device 602 includes second address lines 607 and a second enabling line 608. During each electrode coupling operation, addresses are supplied to the address busses but line selection does not occur until respective enabling signals have been received.

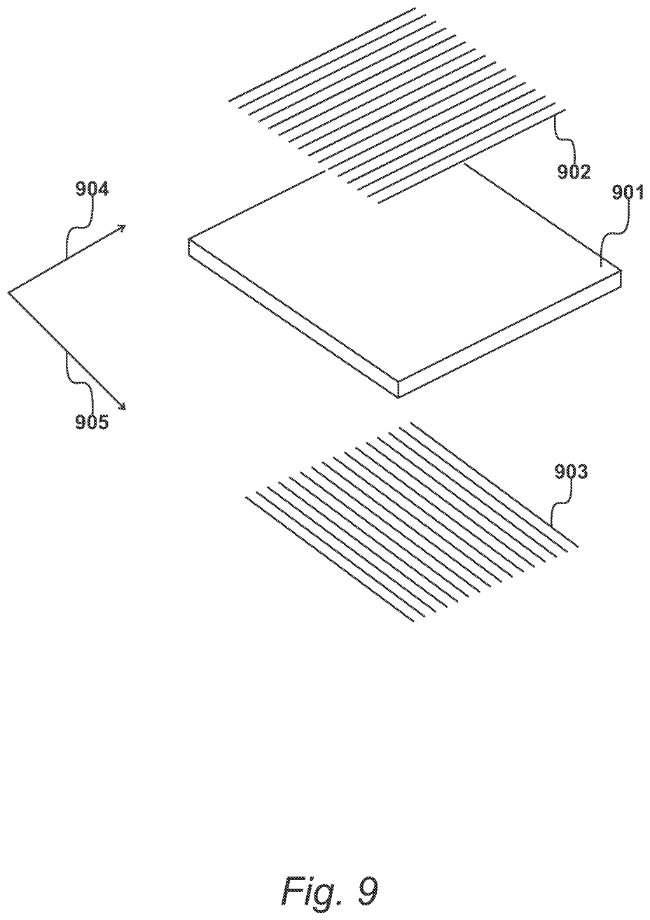

### FIG. 9

The provision of two mutually orthogonal electrode groups is illustrated in FIG. 9. A dielectric membrane 901 is substantially similar to that shown in FIG. 1. The subassembly includes a first group of electrodes 902, along with a second group of electrodes 903. In this example, fifteen electrodes are provided in each group and for each group, the electrodes are substantially linear and substantially parallel. However, the groups are mutually offset and, in this embodiment, arranged in mutually orthogonal orientations.

The first group of electrodes 901 are aligned in an x dimension, illustrated by a first arrow 904 and the second group of electrodes 903 are aligned in a y dimension, as illustrated by a second arrow 705. Layering is achieved by coupling electrodes of a first set (selected from a group) and then repeating a scanning operation by coupling electrodes in a second set, selected from the same group. Thus, layering operations performed by the first group of electrodes 902 achieve a layering operation in the direction of the second arrow 905. Similarly, the second group of electrodes 903 achieve a similar layering operation in the direction of the first arrow 904.

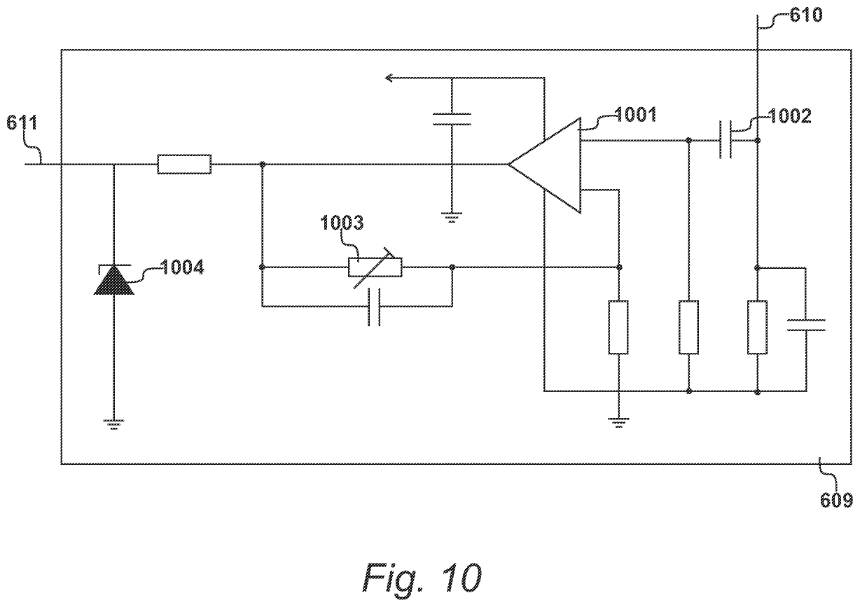

### FIG. 10

Analog processing circuit 609 is shown in FIG. 10. Signals received on the first analog line 610 are supplied to a buffering amplifier 1001 via a decoupling capacitor 1002. During an initial set-up procedure, a variable feedback resistor 1003 is trimmed to optimize the level of monitored signals supplied to the processor 602 via the second monitoring line 411. A Zenner diode 1004 prevents excessive voltages being applied to the processor 402.

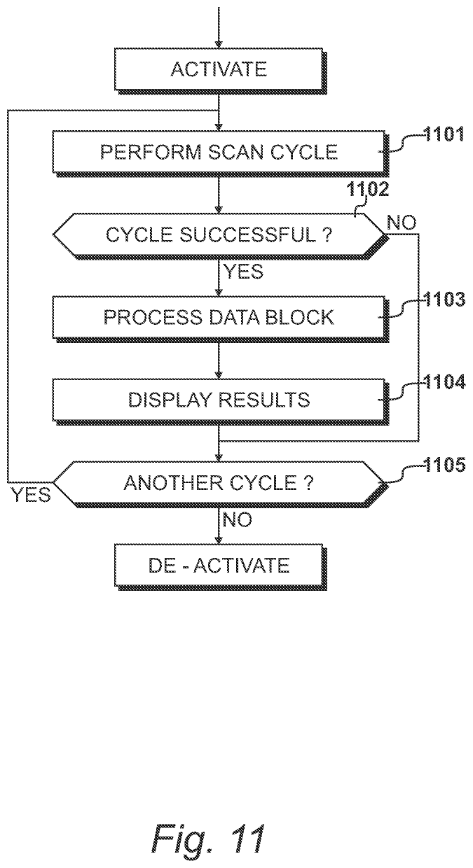

### FIG. 11

Procedures performed by processor 603 are illustrated in FIG. 11. After activation of the apparatus, a scan cycle is performed at step 1101. In an embodiment, this scan cycle consists of first performing calibration procedures, without human body contact, followed by testing procedures during which body contact has been established.

When body contact is made, a detector is configured to produce force data representing an applied force or pressure upon an active surface. The processor is configured to compare the force data against a first predetermined level. Thereafter, a decision is made as to whether it is possible to perform capacitive coupling procedure that capacitively couple selected pairs of the electrodes by producing electric fields that penetrate the tissue to produce monitored output signals, which occurs if the force data is higher then the predetermined level. Alternatively, if the force data is not higher than this predetermined level, capacitive coupling procedures are inhibited.

Thus, as illustrated in FIG. 11, after performing a scan cycle, a question is asked at step 1102 as to whether the cycle has been successful. Thus, if insufficient pressure was applied, the question asked at step 1102 will be answered in the negative. However, if a successful scanning cycle has been achieved, the question asked at step 1102 will be answered in the affirmative, allowing a data block to be processed at step 1103. Thereafter, at step 1104, results may be displayed.

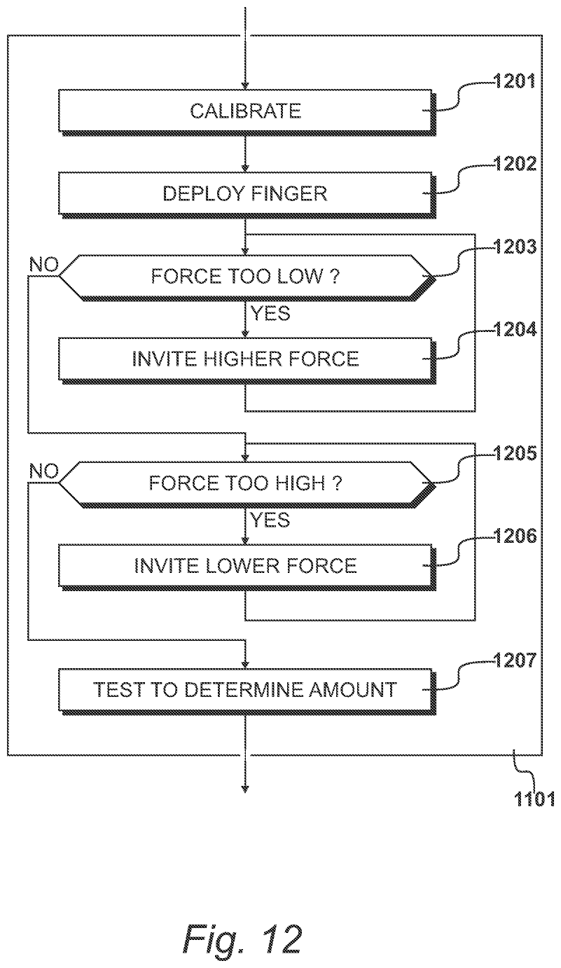

### FIG. 12

Thus, it has been appreciated that it is not possible to obtain satisfactory results if a subject does not apply their finger (or other body art) onto an application region with sufficient force. For satisfactory results to be obtained, a minimum allowable force is established by the first predetermined level. If the measured force does not achieve this level, further capacitive coupling procedures are inhibited.

Further investigations also suggest that problems can arise if a subject applies too much force upon the active surface. Under these conditions, tissue and hence the distribution of blood within capillaries, is distorted, often to an extent where compensation cannot be achieved. Thus, in an embodiment, as illustrated in FIG. 12, the processor is also configured to inhibit capacitive coupling procedures if the force data is above a second predetermined level.

An example of a scan cycle 1101 is illustrated in FIG. 12. At step 1201 calibration procedures are conducted which, in an embodiment, replicate all of the procedures that will be performed during the testing stage. Thus, in the test data set, every data point has an equivalent point in the calibration data set.

After performing the calibration procedures, without a finger being in place, a subject is invited to deploy a finger at step 1202. At step 1203 a question is asked as to whether the applied force is too low. Thus, at this stage, force data is compared against the first predetermined level. If this question is answered in the affirmative, to the effect that insufficient pressure has been applied, the subject is invited to apply higher force at step 1204. Thus, in response to this invitation, the force is examined again at step 1203 and further invitations may be made if insufficient force continues to be applied.

In most situations, the question asked at step 1203 will eventually be answered in the negative, thereby allowing progress to step 1205. However, as is known in the art, the loop created by steps 1203 and 1204 may include timeout provisions, resulting in the generation of an error message if a subject is unable to apply an appropriate level of force.

In response to the question asked at step 1203 being answered in the negative, confirming that a sufficient level of force has been applied, in this embodiment, a question is asked at step 1205 as to whether the applied force is too high. Thus, again, if this question is answered in the affirmative, the subject is invited to apply a lower force at step 1206. Thus, again, the force may be tested at step 1205 and, under most circumstances, the question asked at step 1205 will eventually be answered in the negative such that testing procedures, similar to the calibration procedures, may be performed at step 1207.

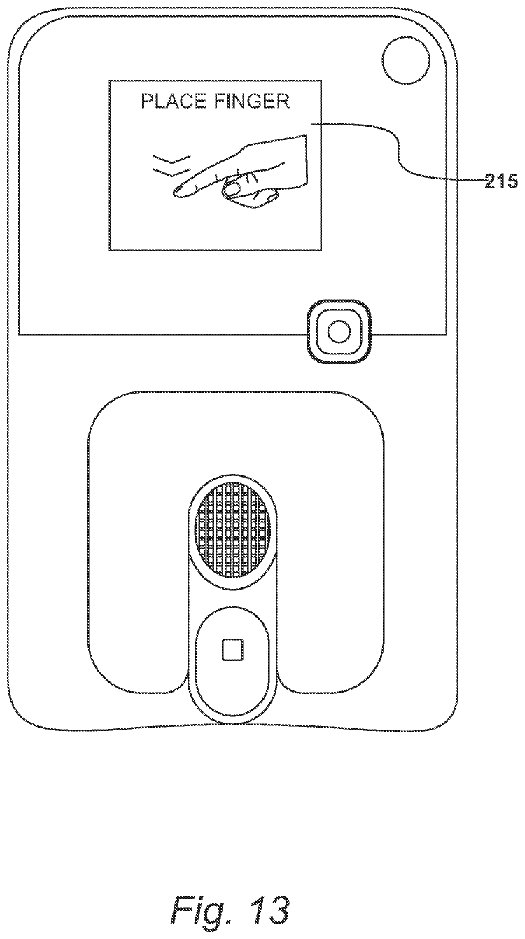

### FIG. 13

The apparatus described herein evaluates an amount of a substance contained within blood circulating within human body tissue. Concentrations of many different substances may be evaluated, provided that they change the dielectric properties of the blood. These include inorganic, organic and bio-chemical substances. Features of the embodiment will be described with reference to an evaluation of glucose levels, given the prevalence of medical conditions requiring this substance to be evaluated regularly.

After performing a calibration procedure, the visual display unit 215 displays a message, along with a graphic, inviting a finger to be placed on the apparatus. Thus, instructions are displayed to a subject on the visual display unit, to assist the subject completing the overall evaluation procedure.

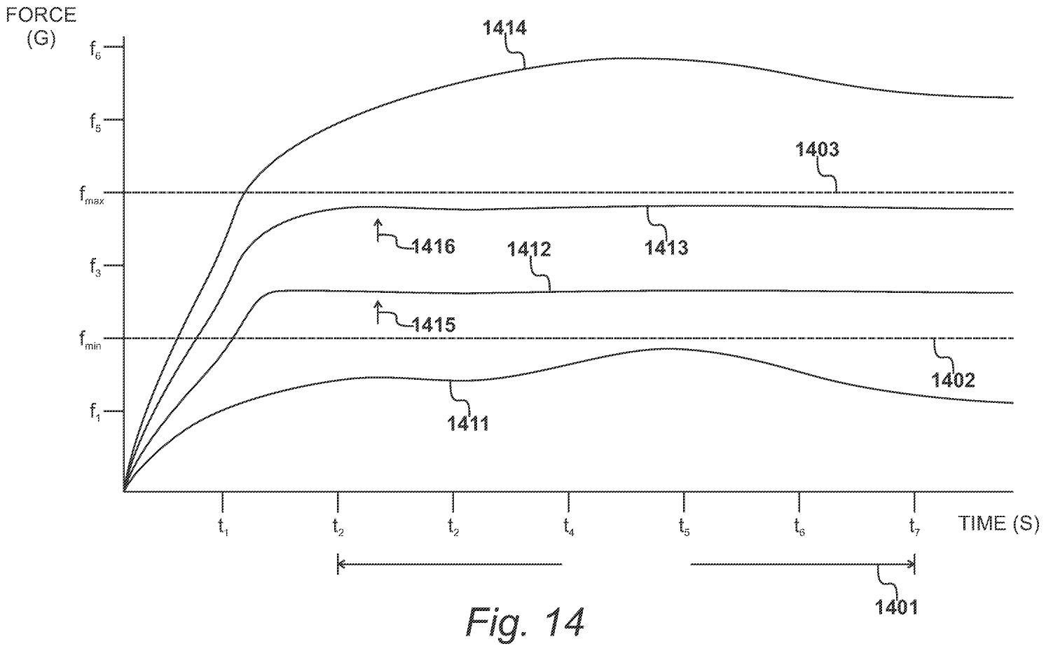

### FIG. 14

The present invention provides a method of evaluating an amount of a substance contained within blood circulating within the human body tissue. An application region of an apparatus is located to make skin contact at the position of the tissue, with the apparatus itself including electrodes arranged on a dielectric substrate to present an active surface at the application region. Force data is derived from a detector, as illustrated in FIG. 14, where applied force is plotted against time. In an embodiment, a subject deploys a finger upon the application region of the apparatus. With a finger in place, evaluation takes place during an evaluation period 1401.

Force data, representing applied force or applied pressure upon the apparatus, is compared against the first predetermined level 1402. In an embodiment, a comparison is also made against a second predetermined level 1403.

For the purposes of illustration, four deployments are illustrated in FIG. 14, consisting of a first deployment 1411, a second deployment 1412, a third deployment 1413 and a fourth deployment 1414.

Referring to deployment 1411, it can be seen that the level of force applied is below the first predetermined level 1402. Thus, under these circumstances, the capacitive coupling procedures are inhibited and in accordance with an embodiment, the subject is invited to apply more pressure.

In the fourth deployment 1414, the applied pressure is greater than the first predetermined level 1402 but, on this occasion, it is also greater than the second predetermined level 1403. Thus, in this embodiment, the capacitive coupling procedures are again inhibited and a subject is invited to repeat the procedure while applying a lower pressure.

For the second deployment 1412 and the third deployment 1413, the level of applied pressure is above the first predetermined level 1402 while being below the second predetermined level 1403. Consequently, capacitive coupling procedures are performed that capacitively couple selected pairs of the electrodes by producing electric fields that penetrate the tissue to produce monitored output data.

Although the second deployment and the third deployment both allow test data to be produced, it should also be appreciated that different levels of force were maintained during period 1401. Experiments have shown that a greater degree of accuracy can be achieved if account is made of this measured force. Thus, in an embodiment, before initiating the capacitive coupling procedures of the test, the applied pressure is actually measured and appropriate data recorded. Thus, for the second deployment 1412 force data is recorded, as indicated by arrow 1415. Similarly, for the third deployment 1413, force data is again recorded as indicated by arrow 1416.

In an alternative embodiment, applied force is measured periodically during the scanning operation to ensure that this has not gone below the first predetermined level or above the second predetermined level. Under these circumstances, further scanning may again be inhibited and a subject invited to start the process again.

In a further embodiment, several pressure measurements may be recorded during the scanning process, even when each result continues to be between the lower threshold and the higher threshold. Thus, a first pressure value may be recorded at the start of layering using the first group of electrodes, with a new reading being taken at the start of layering with the second group of electrodes. In an embodiment, for each group, forward layering is followed by reverse layering and pressure values may be recorded separately for the forward and reverse components.

In an embodiment, temperature data and humidity are also measured and recorded. Temperature may be measured at the position of the finger, by detector 107 and inside the apparatus itself. Thus, all four measurements may be used to compensate measured values to improve overall accuracy. For glucose measurement, the aim is to obtain results that are at least as accurate, and preferably more accurate, than the results obtained using known invasive techniques.

Experiments have also shown that overall accuracy can be improved if the whole procedure is repeated, so that results can be averaged or compared. In an embodiment, three blocks or data are created for each subject by performing all of the procedures three times. If one of the data blocks produces results that are significantly different from the other two, this block is rejected and a selection is based on one of the other two or the other two are averaged.

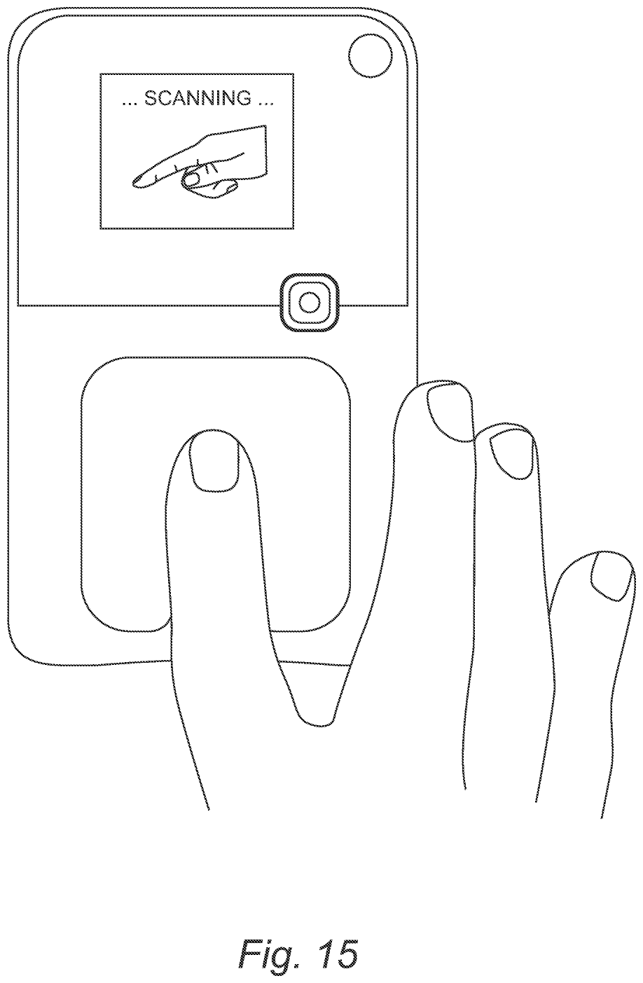

### FIG. 15

After having been invited to deploy a finger, as described with reference to FIG. 13, a finger is engaged against the substantially parallel electrodes protected by the insulating coating. Thus, the electrodes are supported by the main circuit board and are exposed through the first membrane-exposing orifice in the housing.

A control circuit energizes and monitors selected electrodes to produce output data indicative of blood glucose concentrations. The visual display unit confirms this operation by identifying the apparatus as “scanning” as shown in FIG. 15. Furthermore, throughout this procedure, the applied pressure is monitored by means of the force sensor supported by the bottom circuit board and force data is stored by the processor 602 when capacitive coupling procedures are being performed.

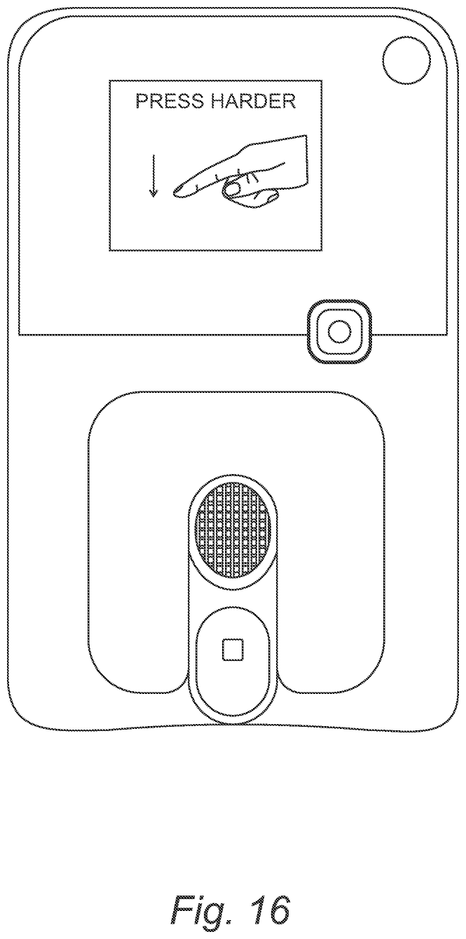

### FIG. 16

As previously described, step 1202 invites a subject to deploy a finger, as described with reference to FIG. 13. An invitation of the type generated at step 1204 is illustrated in FIG. 16. Thus, in response to an evaluation being made to the effect that applied force is too low, the subject, via the visual display 215, is invited to press harder on the detector, so that the procedures may be repeated.

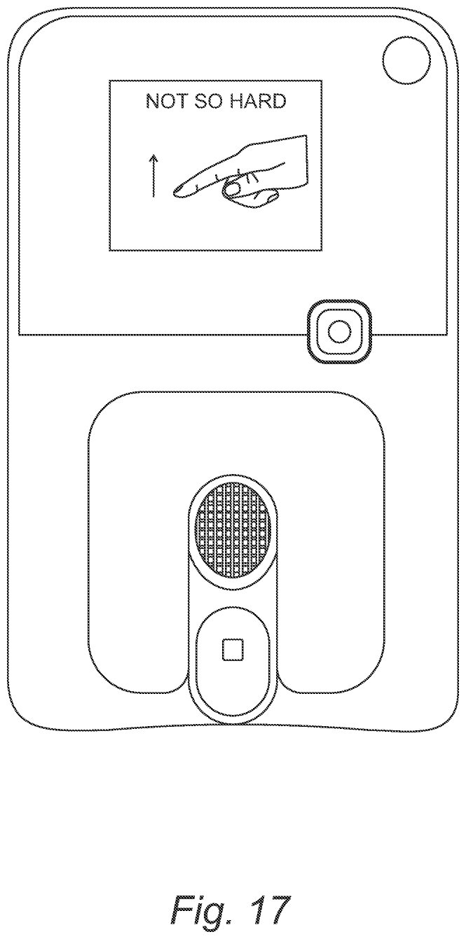

### FIG. 17

At step 1205, a question has been asked as to whether the force was too high and when answered in the affirmative, step 1206 invites a user to deploy lower force. Thus, as illustrated in FIG. 17, the visual display unit 215 invites the subject to press not so hard, such that this will hopefully result in the question asked at step 1205 being answered in the negative, such that test data may be established at step 1207.

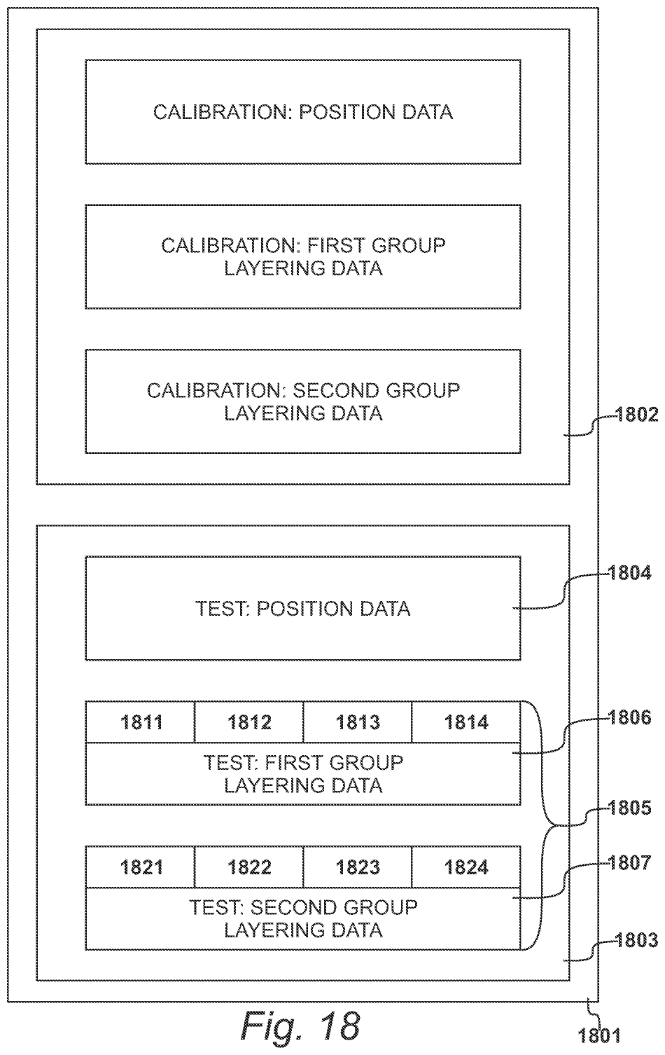

### FIG. 18

A scanning cycle, of the type usually performed for each individual subject, produces an output data block that is processed at step 1103. An example of an output data block 1801 is shown in FIG. 18. Each output data block consists of calibration data 1802 and test data 1803. In this embodiment, the test data 1803 includes test position data 1804 and test layering data 1805.

When two groups of electrodes are present, as described with reference to FIG. 7, the test layering data 1805 comprises a first layering data set 1806 and a second layering data set 1807. As illustrated in FIG. 18, the calibration data also includes a similar data structure.

In this embodiment, the test layering data 1805 also includes the previously described environment data, consisting of force data, finger temperature data, internal device temperature data and humidity data. In this embodiment, respective measurements are recorded prior to first test layering procedures and prior to second test layering procedures. Thus, first group layering data 1806 includes force data 1811, finger temperature data 1812, internal temperature data 1813 and humidity data 1814. Similarly, second group layering data 1807 includes force data 1821, finger temperature data 1822, internal temperature data 1823 and humidity data 1824.

In an alternative embodiment, environment data is measured twice during each layering procedure. Firstly, at the start of forward layering and secondly at the start of reverse layering. Thus, four results are recorded for each data block. A scanning operation may produce a single data block or, in an embodiment, the procedures may be repeated twice to produce three data blocks, resulting in twelve environment data sets being recorded. Environment data may also be recorded during procedures other than layering during the overall scanning process.

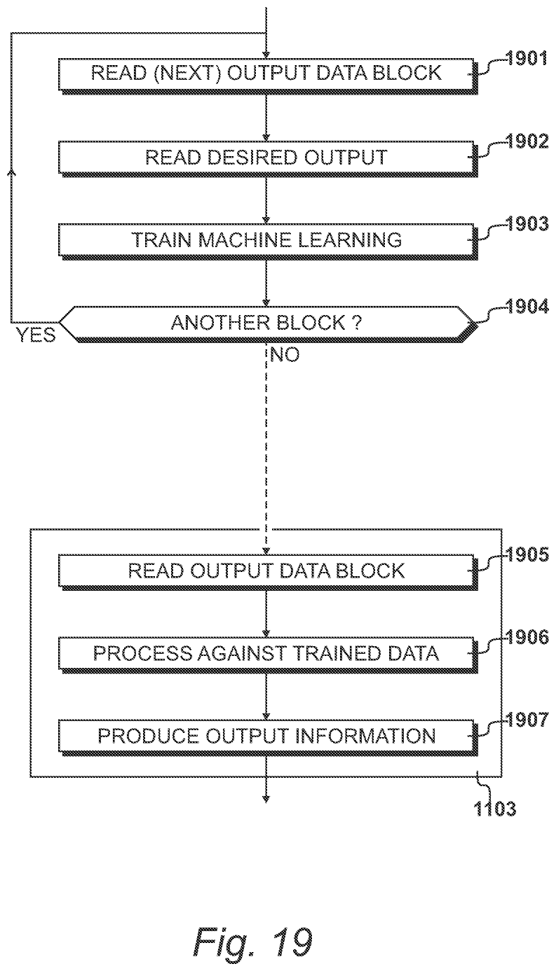

### FIG. 19

In an embodiment, processing step 1103 is performed using a machine learning system. In this embodiment, plural learning output data blocks are produced for a first selection of subjects, for which the extent to which a substance under investigation (such as glucose) is present, is known.

Plural learning output data blocks are deployed to prepare a machine learning system. Live output data blocks are then processed, at step 1103, by means of the machine learning system, to produce respective evaluation data for the substance under investigation.

Machine learning systems of this type deploy regression algorithms to produce continuous outputs which, for example, may identify the level of glucose present within blood capillaries of the finger. In an alternative embodiment, a classification algorithm may be deployed to identify whether, for example, a glucose level is too low, normal of too high.

As is known in the art, each training example is represented by a vector and the training data is presented in a matrix. Through iterative operation of an objective function, supervized learning algorithms learn a function that can be used to predict the output associated with new inputs. Thus, an optimized function allows the algorithm to correctly determine the output for inputs that were not part of the original training data. Furthermore, after conducting a training procedure, the system will have learnt to perform the task required and it is therefore possible to provide an accurate evaluation of the amount of the substance (such as glucose) present in blood of the subject.

Operations performed with respect to the machine learning system are shown in FIG. 19. Steps 1901 to 1904 relate to the training of the system, whereafter steps 1905 to 1908 relate to the deployment of the system at step 1103.

At step 1901, the next output data block, having a structure of the type described with reference to FIG. 18, is read and the related desired output is then read at step 1902. Thus, during the training operation, it is necessary to make measurements using alternative procedures. Thus, for example, when training a glucose monitoring apparatus, glucose measurements are made using conventional invasive techniques.

At step 1903, the machine learning system is trained in response to the data received at step 1901 and step 1902, whereafter at step 1904, a question is asked as to whether another block is to be considered. When answered in the affirmative, the next output data block is read at step 1901.

As is known in the art, the accuracy of the system will improve with the number of training iterations that are possible and this will be dependent upon the availability of data. During this process, random tests can be conducted to determine the accuracy of the system and a convergence towards accurate results should be witnessed. Only when an appropriate convergence has been achieved is it then possible to progress to the next stage.

Thus, the next stage represents procedures performed at step 1103. Again, an output data block, of the type described with reference to FIG. 18, is read at step 1905. At step 1906, this data block is processed against the trained data produced by the procedures described above. Thereafter, at step 1907, output information is produced and from this, results are displayed at step 1104.

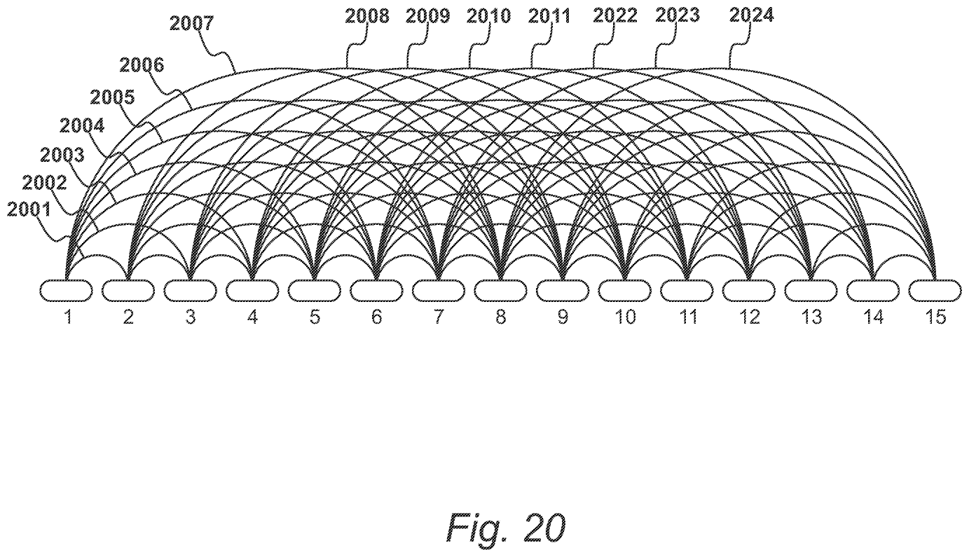

### FIG. 20

The electrodes of the first group 902 are shown in FIG. 20, numbered 1 to 15. Electronics within the evaluation apparatus, as described with reference to FIG. 6, generates energization pulses for application to any of the electrodes 1 to 15 as a transmitter electrode. In addition, output signals may be monitored from any remaining one of the electrodes as a receiver electrode, wherein a peak value of an output signal is indicative of permittivity and a decay rate of an output signal is indicative of conductively. Thus, during each energization operation, an energized transmitter electrode and a monitored receiver electrode define a capacitively coupled electrode pair.

From the available electrodes 1 to 15 of the selected group, a first set of n electrodes is selected. Thus, in the example shown in FIG. 20, all fifteen electrodes are selected as the first set of n electrodes. However, it is not necessary for all of the available electrodes to be selected in this way, such that, in alternative embodiments, the selected set may contain fewer electrodes than the selected group.

Capacitively coupled electrode pairs are established, in which each of the first set of n electrodes is capacitively coupled with a second set of m electrodes selected from the first set of n electrodes. Thus, the second set of m electrodes is a subset of the first set of n electrodes. Thus, for each selected electrode of the first set, a respective second set of m electrodes is identified. These m electrodes are capacitively coupled with the selected electrode of the first set.

In an embodiment, this capacitive coupling may occur in parallel, requiring multiple analog to digital conversion devices operating in parallel. However, in this embodiment, capacitive coupling occurs sequentially such that, at any instant, only one electrode of the first set is coupled with only one electrode of the second set. To achieve this coupling, either electrode may be energized as a transmitter, with the other electrode of the pair being monitored as the receiver.

Furthermore, in this embodiment, each second set of m electrodes are the nearest neighbouring electrodes to an electrode selected from the first set of n electrodes. Consequently, the number of electrodes present in the second set of m electrodes represents a degree of layering.

Following this method, all of the resulting electric fields are illustrated in FIG. 20. The first set of n electrodes consists of all fifteen available electrodes within the group. The degree of layering is seven, as illustrated by electric fields 2001 to 2007. Each of the first set of n electrodes is capacitively coupled with a second set of n electrodes. Thus, when considering the first electrode 1 as being a member of the first set, it is capacitively coupled with electrodes 2 to 8. Electrode 1 is not capacitively coupled with electrodes 9 to 15. Thus, the second set of m electrodes (2 to 8) with reference to the first electrode 1, are the nearest neighbouring electrodes to electrode 1, selected from the remaining n electrodes of the first set. Thus, having achieved a seventh degree of layering, it would be necessary to couple electrode 1 with electrode 9, should an eighth degree of layering be required.

The selected electrodes may be capacitively coupled in any order, provided that all of the electric fields illustrated in FIG. 20 are realized.

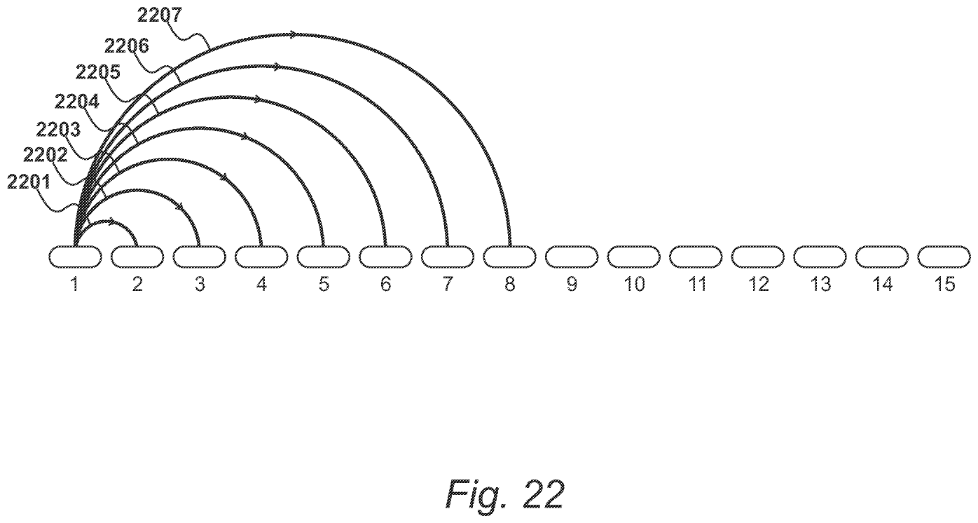

Previous investigations have identified advantages with respect to having multiple couplings with the end electrodes which, in this example, are identified as electrode 1 and electrode 15. Such an approach provides useful layering data. However, in the present embodiment, many more layering opportunities are established through a dynamic process. The embodiments described herein develop these techniques to collect the required data in a systematic way and this approach will be described with reference to FIGS. 22 to 30. However, it should be appreciated that other approaches may be adopted to achieve the result of deriving data from coupling m electrodes that are the nearest neighbouring electrodes to an electrode selected from the first set of n electrodes.

As previously described, data derived from the seventh degree of layering is achieved by electric field 2007, from the coupling of electrode 1 with electrode 8. Similar seventh degree fields are shown at 2008 (coupling electrodes 2 and 9), 2009 (coupling electrodes 3 and 10), 2010 (coupling electrodes 4 and 11), 2011 (coupling electrodes 5 and 12), 2012 (coupling electrodes 6 and 13), 2013 coupling electrodes 7 and 14) and 2014 (coupling electrodes 8 and 15).

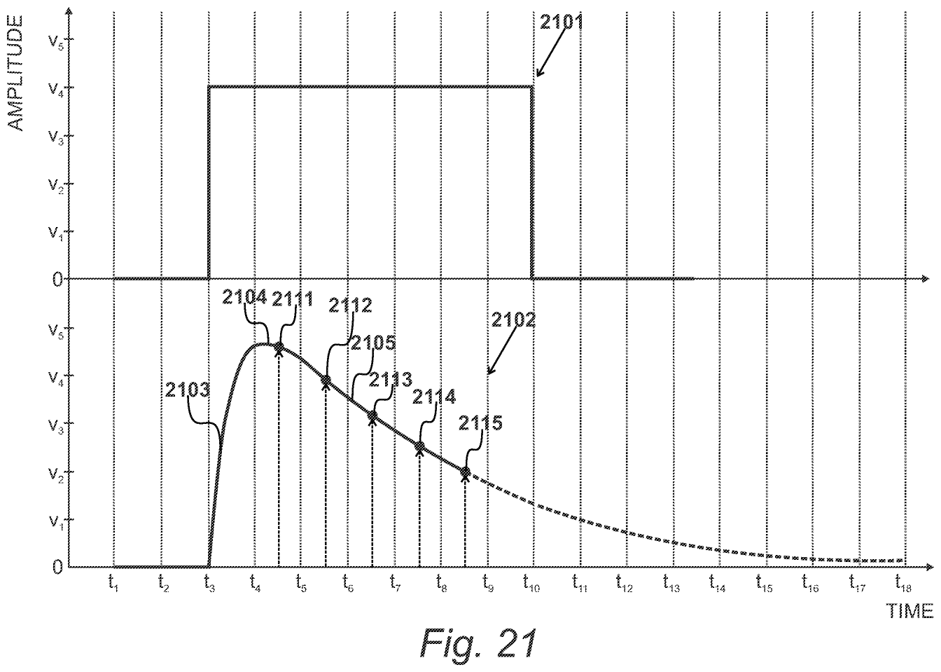

### FIG. 21

It can be appreciated that to achieve dynamic layering, a significant number of coupling operations are required for each scanning procedure, as described with reference to FIG. 20. Furthermore, in an embodiment, each output signal produced from each capacitively coupled electrode pair is sampled to produce a coupling data set, in which a first sample of each coupling data set is indicative of permittivity and subsequent samples of each coupling data set are indicative of conductivity.

An energizing input pulse 2101 is shown in FIG. 21, plotted on a shared time axis with a monitored output signal 2102. The energizing input pulse 2101 rises relatively sharply to increase the high frequency components and hence produce a high-frequency-dependent response characteristic. Thus, in this embodiment, a frequency response is achieved without being required to sweep through many sinusoids of different frequencies.

The monitored output signal 2102 has been amplified by the circuit described with reference to FIG. 10 and is then sampled by an analog-to-digital convertor forming part of the processor 602. The monitored output signal 2102 has a rising edge 2103, a peak 2104 and a falling edge 2105. The peak level 2104 will be determined predominantly by permittivity characteristics. Similarly, the falling edge 2105 represents a decay of the induced field and the rate of decay will be determined predominantly by conductivity characteristics. Thus, by recording multiple samples, it is possible to obtain rich coupling data sets. To achieve this, as illustrated in FIG. 21, a first sampling point 2111 is followed by a second sampling point 2112, followed by a third sampling point 2113, a fourth sampling point 2114 and a fifth sampling point 2115.

The processor 602 is responsible for initiating the energization input signal, therefore the processor is instructed with an appropriate delay period before initiating the sampling process. This delay is determined empirically and aims to place the first sampling point at the peak value. However, a degree of tolerance is permitted, as illustrated in FIG. 21, given that the same delay period is used for each coupling operation, allowing comparisons to be made between similar examples. However, for the purposes of this embodiment, it should be appreciated that each coupling operation results in the generation of an output signal substantially similar to that shown at 2102, which in turn generates a coupling data set containing five data points. In other embodiments, more or fewer data samples may be recorded.

### FIG. 22

The present embodiment is configured to capacitively couple electrodes to establish the pattern shown in FIG. 20. The actual order for doing this is not relevant but the present embodiment takes a systematic approach to facilitate the programming of processor 602. In particular, in an embodiment, capacitively coupled electrode pairs are established by sequentially selecting each of the n electrodes of the first set as an electrode in common. Furthermore, for each selected electrode in common, the procedure sequentially defines capacitively coupled electrode pairs with the second set of m nearest neighbouring electrodes. Thus, as illustrated in FIG. 15, a procedure may start by selecting electrode 1 as the first electrode in common and then sequentially capacitively coupling electrode 1 with the m nearest neigbouring electrodes, which are electrodes 2 to 8, when layering to the seventh degree is required.

To achieve the pattern shown in FIG. 22, the electrode in common (electrode 1) could be selected as a transmitter electrode or as a receiver electrode. In this embodiment, electrode 1 is selected as a transmitter electrode for each coupling operation, as represented by the arrow on each of the field lines. Thus, in an embodiment, the procedure is initiated by energizing electrode 1 and monitoring electrode 2 to produce a first electric field 2201. Electrode 1, as the electrode in common, is energized again but with electrode 3 being monitored, to produce a second electric field 2202. Again, as the electrode in common, electrode 1 is energized and a third electric field 2203 is produced by monitoring electrode 4. This process is repeated with respect to electrode 5 being monitored, electrode 6 being monitored, electrode 7 being monitored and electrode 8 being monitored, producing respective electric fields 2204 to 2207.

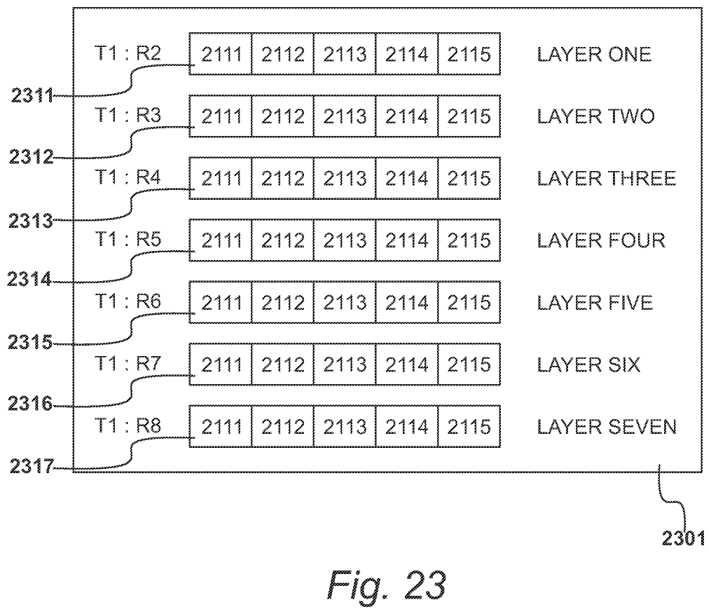

### FIG. 23

The procedure described with reference to FIG. 22 produces a common electrode data set 2301. This is made up of seven coupling data sets 2311 to 2317, from the first transmitter electrode 1 coupling with seven receiver electrodes 2 to 8. In addition, as the distance between the electrodes increases, the degree of layering also increases. Thus, the first coupling data set 2311 may be identified as belonging to layer one, with the second belonging to layer two, the third belonging to layer three and so on, with the seventh 2317 belonging to layer seven.

As described with reference to FIG. 21, each coupling data set consists of five data samples 2111 to 2115.

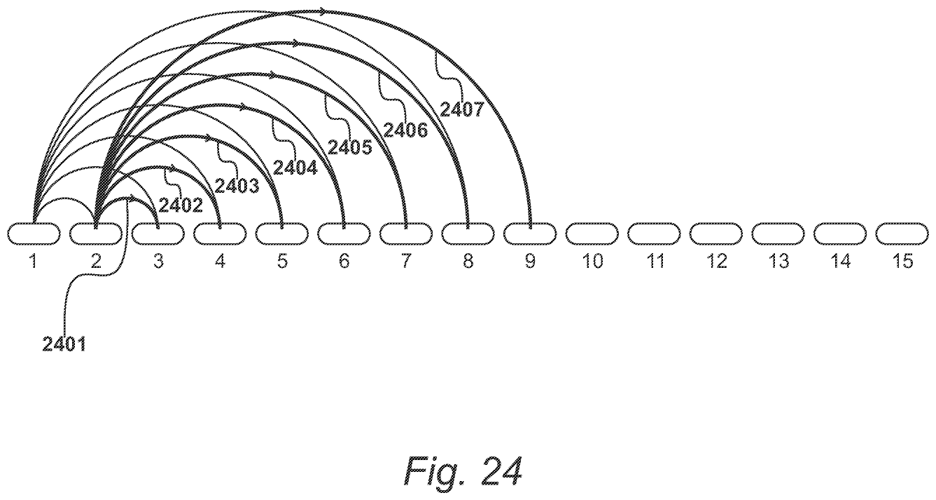

### FIG. 24

In this embodiment, the systematic selection of electrodes started by selecting a first end electrode, electrode 1, as an electrode in common to produce the first common electrode data set 2301. The process continues by sequentially selecting adjacent electrodes as electrodes in common in a first direction of (forward) dynamic layering, until a second end electrode (electrode 15 in this example) is reached.

Thus, in accordance with this embodiment, having selected electrode 1 as the electrode in common, adjacent electrode 2 is now selected as the electrode in common, resulting in the generation of electric fields 2401 to 2407.

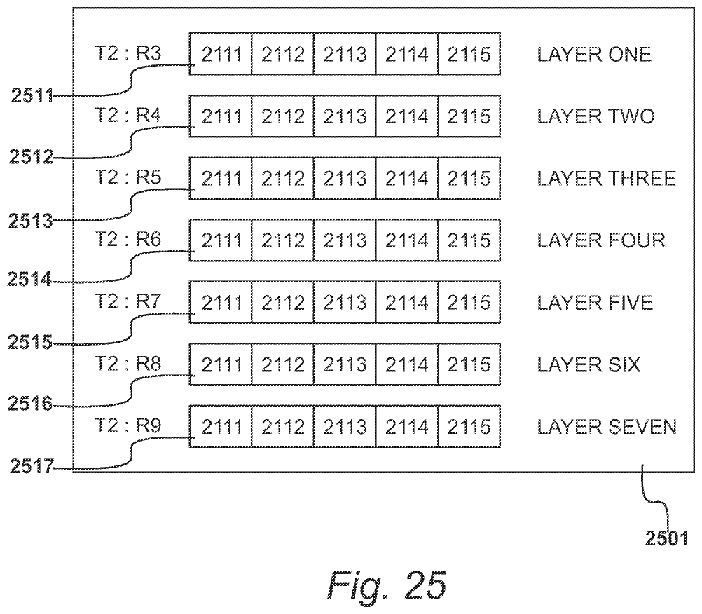

### FIG. 25

The coupling operations illustrated in FIG. 24 result in the production of a second common electrode data set 2501. Again, this produces a further seven coupling data sets 2511 to 2517, representing the seven layers of penetration, each again including five coupling data sets 2101 to 2105.

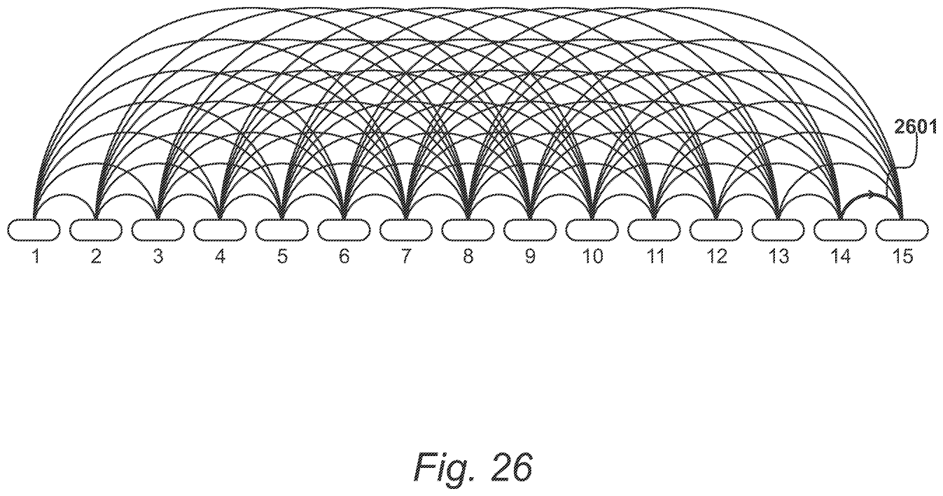

### FIG. 26

The process of sequentially selecting adjacent electrodes as electrodes in common continues until a second end electrode is reached, as illustrated in FIG. 19. When electrode 8 is selected as the electrode in common, a full seven layers of penetration can be achieved, giving the couplings that are possible with electrodes 9 to 15. However, as subsequent electrodes are selected as the electrode in common, the level of penetration reduces until, as shown in FIG. 19, when electrode 14 is selected as the electrode in common, it is only possible for it to couple with electrode 15, as illustrated by electric field 2601.

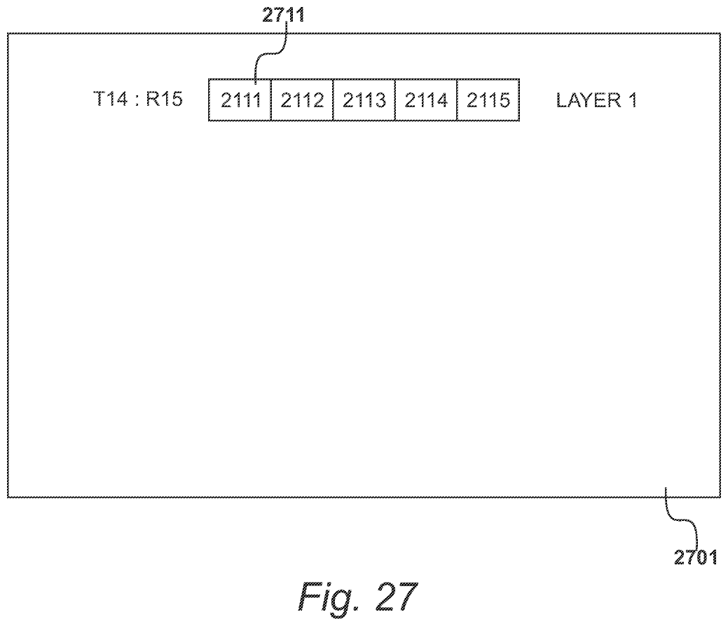

### FIG. 27

As illustrated in FIG. 27, when electrode 14 is selected as the electrode in common, only one common electrode data set 2701 is produced. Again, this common electrode data set consists of five coupling data sets 2111 to 2115.

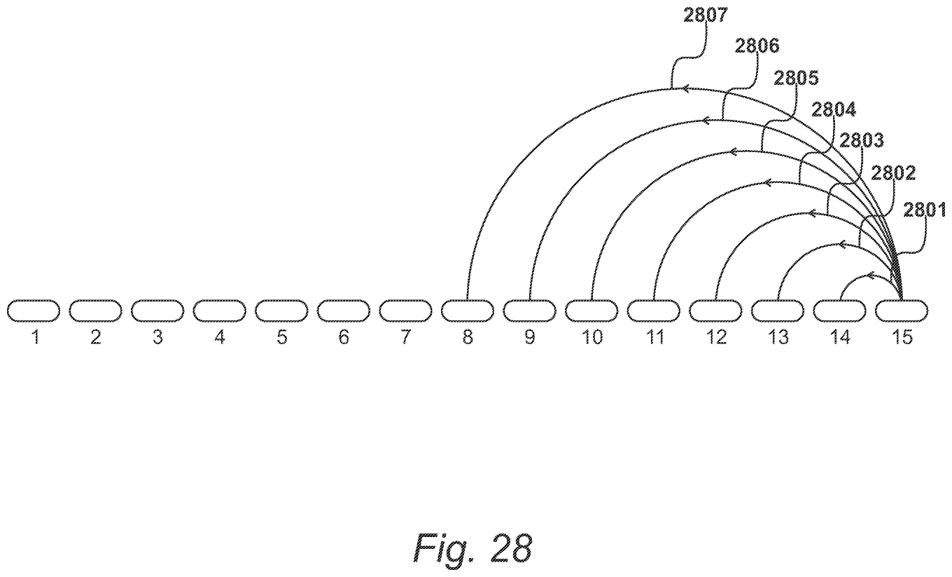

### FIG. 28

After reaching the second end electrode, as described with reference to FIG. 19, the second end electrode 15 is now selected as an electrode in common, as shown in FIG. 28. This results in the sequential generation of electric fields 2801 to 2807.

Thereafter, adjacent electrodes, starting from electrode 14, are sequentially selected as the electrode in common, moving in the second direction of dynamic layering (reverse layering) until the first end (electrode 1) is reached. Thus, for each selected electrode in common, such as electrode 15 in FIG. 28, a second set of m electrodes are selected that are the nearest neighbours (14 to 8), but only in the direction of (reverse) dynamic layering.

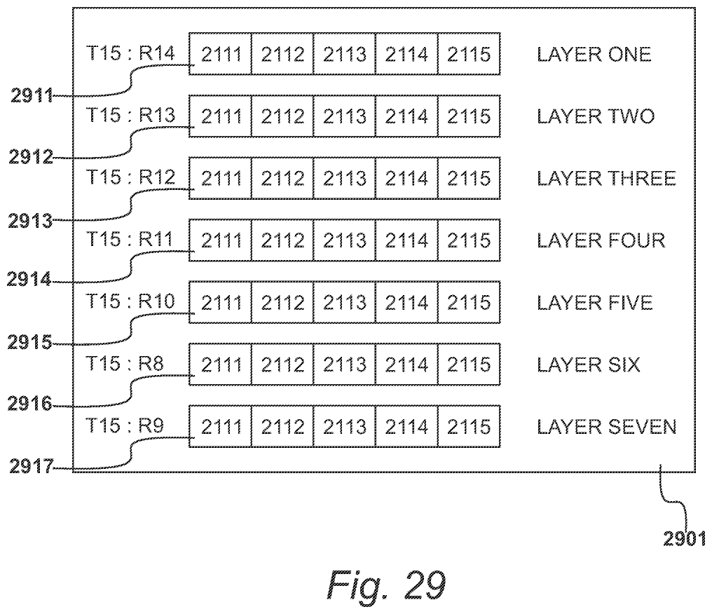

### FIG. 29

The procedures described with reference to FIG. 28 produce a new common electrode data set 2201. Again, on this occasion, penetration is possible to level seven, resulting in the generation of seven coupling data sets 2911 to 2917.

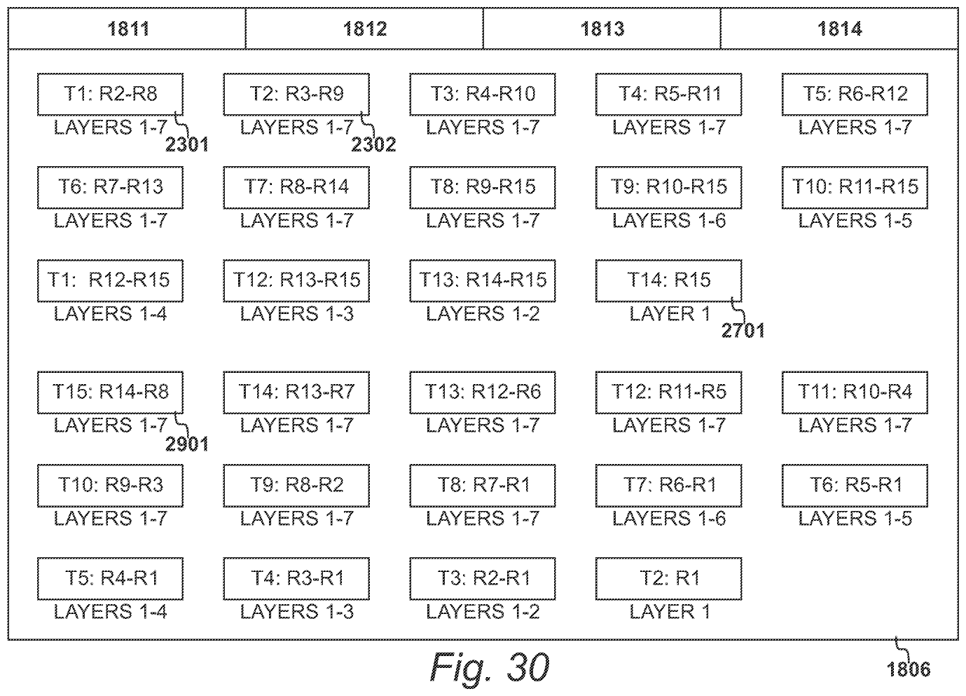

### FIG. 30

As illustrated in FIG. 30, the procedure of forward dynamic layering followed by reverse dynamic layering produces twenty-eight common electrode data sets, which include common electrode data sets 2301, 2302, 2701 and 2201, along with all the others making up a complete layering data set 2901. Furthermore, in this embodiment, the procure also records force data 1811, finger temperature data 1812, internal temperature data 1813 and humidity data 1814. In an alternative embodiment, this environment data is recorded separately for forward layering and reverse layering.

The result, as shown in FIG. 30, is the production of the first electrode group layering data. For a complete scan, to produce the output data block 1001, similar procedures are also performed for the calibration first group layering data, the calibration second group layering data and the test second group layering data 1007.

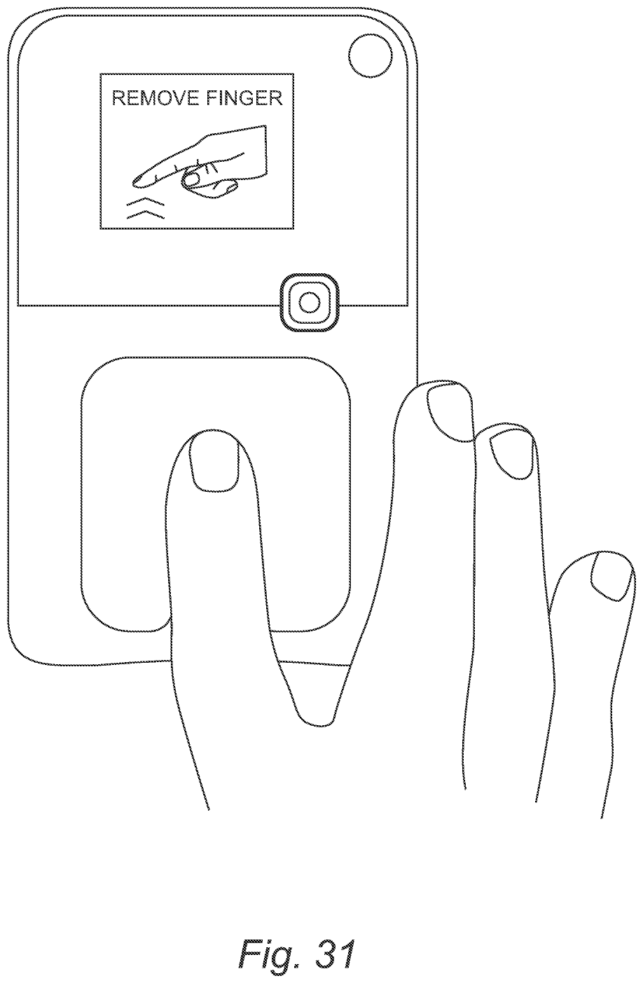

### FIG. 31

After the measuring process has completed, the visual display unit invites the subject to remove their finger, as illustrated in FIG. 8. After removal, the method continues with the step of allowing the elastomeric material to expand, thereby returning the intermediate circuit board to its original position.

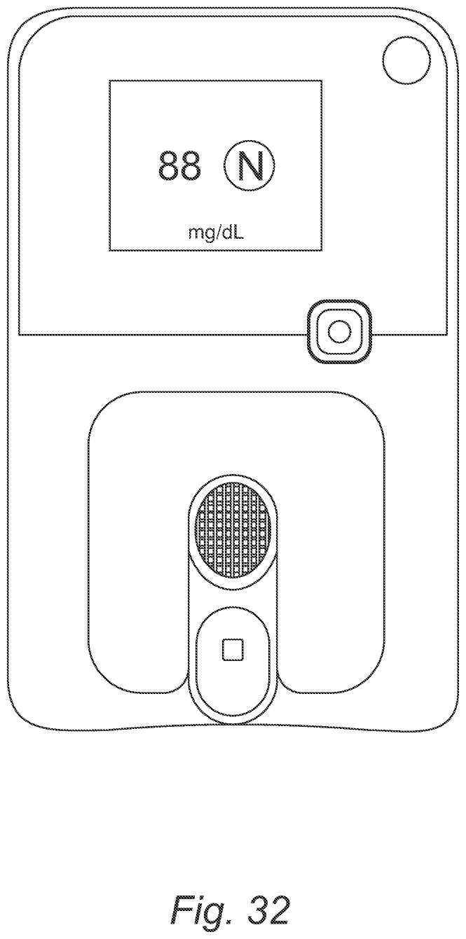

### FIG. 32

After analyzing output data received during the scanning procedure, it is possible for the visual display unit 215 to provide an indication of glucose concentration. Furthermore, in addition to providing a numerical value (representing eighty-eight milligrams of glucose per deci-liter of blood in this example), an indication may also be provided as to whether this concentration is considered to be low, normal or high. In an embodiment, for each of these possibilities, an appropriate colour is displayed. Thus, a low value may be presented in blue, a normal value in green and a high value in red.

It should be appreciated that similar graphical displays may be generated when evaluating concentrations of other blood containing substances.

What we claim is:

1. An apparatus for the non-invasive evaluation of an amount of a substance contained within blood circulating within human body tissue, comprising:
an application region arranged to make skin contact;
a plurality of electrodes arranged on a dielectric substrate, thereby presenting an active surface at the position of said application region;
a detector configured to produce force data representing an applied force or pressure upon said active surface; and
a processor, wherein said processor is configured to:
compare said force data against a first predetermined level;
perform capacitive-coupling procedures that capacitively couple selected pairs of said electrodes by producing electric fields that penetrate said tissue to produce monitored output data, if said force data is higher than said first predetermined level; and
inhibit said capacitive-coupling procedures if said force data is not higher than said first predetermined level.

2. The apparatus of claim 1, comprising a display device configured to indicate that an increase in applied force or pressure is required if said capacitive coupling procedures have been inhibited.

3. The apparatus of claim 1, wherein said processor is also configured to inhibit said capacitive coupling procedures if said force data is higher than a second predetermined level.

4. The apparatus of claim 3, comprising a display device configured to indicate that a reduction in applied force or pressure is required.

5. The apparatus of claim 1, wherein said processor is also configured to store said force data when said capacitive coupling procedures are performed.

6. The apparatus of claim 1, wherein said plurality of electrodes are a substantially parallel first group of electrodes.

7. The apparatus of claim 6, further comprising a second group of substantially parallel electrodes, wherein:
said second group of electrodes is electrically isolated from said first group of electrodes; and
the orientation of said second group of electrodes is offset with respect to the orientation of said first group of electrodes.

8. The apparatus of claim 6, further comprising multiplexing devices configured to:
select any of said electrodes as a transmitter; and
select any remaining electrode of said electrodes as a receiver.

9. The apparatus of claim 8, further comprising an energizing circuit for energizing a selected transmitter electrode, wherein:
the degree of energization is related to the distance between said selected transmitter electrode and a selected receiver electrode during a capacitive-coupling operation; and
each said capacitive-coupling procedure comprises a plurality of capacitive-coupling operations.

10. The apparatus of claim 1, further comprising sampling means for sampling each monitored output signal received from a receiver electrode during each coupling operation, wherein:
each said capacitive-coupling procedure comprises a plurality of capacitive-coupling operations; and
said sampling means is configured to produce a plurality of data samples of each said monitored output signal.

11. A method of determining an amount of a substance contained within blood circulating within human body tissue, comprising the steps of:
locating an application region of an apparatus to make skin contact at the position of said tissue, wherein said apparatus comprises a plurality of electrodes arranged on a dielectric substrate to present an active surface at said application region;
deriving force data from a detector;
comparing said force data, representing applied force or pressure of said skin contact upon said application region, against a first predetermined level; and
in response to said comparing step, performing a step selected from a list consisting of:
performing capacitive-coupling procedures that capacitively couple selected pairs of said electrodes by producing electric fields that penetrate said tissue to produce monitored output data, if sufficient force has been applied; and
inhibiting said capacitive-coupling procedures if said sufficient force has not been applied, wherein
the amount of said substance is evaluated from said monitored output data when said monitored output data is produced.

12. The method of claim 11, further comprising the step of indicating that an increase in applied force or pressure is required if said sufficient force has not been applied.

13. The method of claim 11, further comprising the steps of:
comparing said force data against a second predetermined level; and
inhibiting said capacitive coupling procedures if too much force has been applied.

14. The method of claim 13, further comprising the step of indicating that a reduction of applied force or pressure is required, if said force data is higher than said second predetermined threshold.

15. The method of claim 11, further comprising the step of storing said force data when said capacitive coupling procedures are performed.

16. The method of claim 15, wherein said step of performing capacitive-coupling procedures, comprises the steps of:
performing a plurality of procedure types; and
storing respective force data for each said procedure type.

17. The method of claim 16, wherein said procedure types comprise calibration procedure types, in which capacitive-coupling procedures are performed prior to said step of making skin contact.

18. The method of claim 11, wherein said plurality of electrodes are substantially parallel, and further comprising the steps of:
generating energization pulses for application to any of said electrodes as a transmitter electrode;
monitoring output signals from any remaining one of said electrodes as a receiver electrode, wherein a peak value of an output signal is indicative of permittivity and a decay rate of an output signal is indicative of conductivity, such that during each energization operation, an energized transmitter electrode and a monitored receiver electrode define a capacitively-coupled electrode pair;
selecting a first set of n electrodes from said plurality of substantially parallel electrodes;
establishing capacitively coupled electrode pairs, in which each of said first set of n electrodes is capacitively coupled with a second set of m electrodes from said plurality of substantially parallel electrodes; wherein:
each said second set of m electrodes are the nearest neighbouring electrodes to an electrode selected from said first set of n electrodes; and
the number of electrodes present in said second set of m electrodes represents a degree of layering.

19. The method of claim 18, wherein said step of establishing capacitively coupled electrode pairs comprises the steps of:
sequentially selecting each said n electrode of said first set as an electrode in common; and
for each said selected electrode in common, sequentially defining capacitively-coupled electrode pairs with a second set of m nearest neigbouring electrodes.

20. The method of claim 11, further comprising the steps of:
producing plural learning output data sets for a first group of subjects, for which the amount of a substance under investigation is known, based on permittivity and resistivity properties;
deploying said plural learning output data sets to prepare a machine-learning system; and
analyzing live output data sets by means of said machine-learning system to produce respective amount data for said substance.
